(19) United States
(12) Patent Application Publication
(10) Pub. No.: US 2009/0156826 A1
(43) Pub. Date: Jun. 18, 2009

(54) METHODS FOR THE PREPARATION OF HYDROXY-SUBSTITUTED ARYL SULFAMIDE COMPOUNDS

(75) Inventors: Maria Papamichelakis, Montreal (CA); Jacqueline Francesca Lunetta, Pierrefonds (CA); Mark Lankau, Dollard Des Ormeaux (CA); Luc Richard, Laval (CA); Christopher Kendall, Verdun (CA); Marcelo Cesar Saraiva, Saint Laurent (CA); Xianghui Wen, St-Laurent (CA); Mahmoud Mirmehrabi, Laval (CA); Valerie Paquet, Montreal (CA); Sylvain Daigneault, Laval (CA); Puwen Zhang, Audobon, PA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/332,400

(22) Filed: Dec. 11, 2008

Related U.S. Application Data
(60) Provisional application No. 61/013,200, filed on Dec. 12, 2007.

(51) Int. Cl. C07D 417/06

(52) U.S. Cl. 548/126

(57) ABSTRACT

The present invention is directed to processes for the preparation of hydroxy-substituted aryl sulfamide derivatives of the formula I or pharmaceutically acceptable salts, stereoisomers or tautomers thereof, which are monoamine reuptake inhibitors wherein the constituent variables are as defined herein.

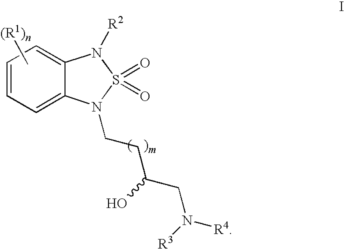

### FIELD OF THE INVENTION

The present invention relates to hydroxy-substituted aryl sulfamide derivatives and precursors thereto, which are monoamine reuptake inhibitors, compositions containing these derivatives, and methods of their preparation.

### BACKGROUND OF THE INVENTION

Compounds described in WO 2008/073459 published Jun. 19, 2008 (hereby incorporated by reference in its entirety) are monoamine reuptake inhibitors for the treatment of conditions, including, inter alia, vasomotor symptoms (such as hot flush), sexual dysfunction (such as desire-related or arousal-related dysfunction), gastrointestinal disorders and genitourinary disorder (such as stress incontinence or urge incontinence), chronic fatigue syndrome, fibromyalgia syndrome, depression disorders (such as major depressive disorder, generalized anxiety disorder, panic disorder, attention deficit disorder with or without hyperactivity, sleep disturbance, and social phobia), diabetic neuropathy, pain, and combinations thereof.

Despite the exploration of a variety of chemistries to provide therapies based on these monoamine reuptake inhibitors, a continuing need exists for preparations, which are efficient and amenable to large-scale syntheses. A need also exists for preparations, which provide compounds free of impurities and any potentially harmful side-products.

### SUMMARY OF THE INVENTION

The present invention is directed to aryl sulfamide derivatives, which are monoamine reuptake inhibitors, compositions containing these derivatives, and processes for their preparation.

One aspect of the invention provides a process for the preparation of a compound of formula I:

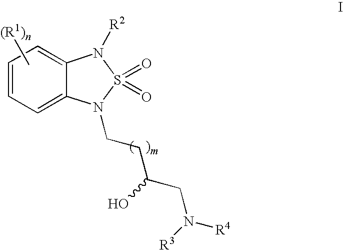

or a tautomer or pharmaceutically acceptable salt thereof;
wherein:
m is an integer from 1 to 3;
n is an integer from 0 to 4;
R¹ is, independently at each occurrence, C₁-C₆alkyl, C₁-C₆alkoxy, halo, CF₃, OCF₃, hydroxy, C₁-C₅alkylC(O)O—, nitro, —CN, C₂-C₆alkenyl, C₂-C₆alkynyl, C₆-C₁₀aryl, C₄-C₁₀heteroaryl, C₁-C₆alkylS(O)—, C₁-C₆alkylS(O)₂—, C₁-C₆alkylS(O)₂NH—, C₁-C₆alkylS(O)₂N(C₁-C₆alkyl)-, C₆-C₁₀arylS(O)₂NH—, C₆-C₁₀arylS(O)₂N(C₁-C₆alkyl)-, C₁-C₅alkylC(O)NH—, C₁-C₅alkylC(O)N(C₁-C₆alkyl)-, or C₆-C₁₀arylC(O)NH—, C₆-C₁₀arylC(O)N(C₁-C₆alkyl)-; wherein each C₆-C₁₀aryl or C₄-C₁₀heteroaryl is independently substituted with 0-3 C₁-C₆alkyl, C₁-C₆alkoxy, halo, CF₃, OCF₃, hydroxy, C₁-C₅alkylC(O)O—, nitro, —CN, C₂-C₆alkenyl, or C₂-C₆alkynyl groups; and each C₆-C₁₀arylS(O)₂NH—, C₆-C₁₀arylS(O)₂N(C₁-C₆alkyl)-, or C₆-C₁₀arylC(O)NH—, C₆-C₁₀arylC(O)N(C₁-C₆alkyl)- is independently substituted with 0-3 C₁-C₆alkyl, C₁-C₆alkoxy, halo, CF₃, OCF₃, hydroxy, C₁-C₅alkylC(O)O—, nitro, —CN, C₂-C₆alkenyl, C₂-C₆alkynyl, C₁-C₆alkylS(O)—, C₁-C₆alkylS(O)₂—, C₁-C₆alkylS(O)₂NH—, C₁-C₆alkylS(O)₂N(C₁-C₆alkyl)-, C₁-C₅alkylC(O)NH—, or C₁-C₅alkylC(O)N(C₁-C₆alkyl)- groups;
R² is C₆-C₁₀aryl or C₄-C₁₀heteroaryl substituted with 0-5 C₁-C₆alkyl, C₁-C₆alkoxy, halo, CF₃, OCF₃, hydroxy, C₁-C₅alkylC(O)O—, nitro, —CN, C₂-C₆alkenyl, C₂-C₆alkynyl, C₁-C₆alkylS(O)—, C₁-C₆alkylS(O)₂—, C₁-C₆alkylS(O)₂NH—, C₁-C₆alkylS(O)₂N(C₁-C₆alkyl)-, C₆-C₁₀arylS(O)₂NH—, C₆-C₁₀arylS(O)₂N(C₁-C₆alkyl)-, C₁-C₅alkylC(O)NH—, C₁-C₅alkylC(O)N(C₁-C₆alkyl)-, C₆-C₁₀arylC(O)NH—, C₆-C₁₀arylC(O)N(C₁-C₆alkyl)-, or C₆-C₁₀aryl or C₄-C₁₀heteroaryl optionally substituted with C₁-C₆alkyl, C₁-C₆alkoxy, halo, CF₃, OCF₃, hydroxy, C₁-C₅alkylC(O)O—, nitro, —CN, C₂-C₆alkenyl, or C₂-C₆alkynyl;
R³ and R⁴ are, independently, H, C₁-C₆alkyl, C₇-C₁₆arylalkyl or (C₄-C₁₀heteroaryl)methyl, wherein each of C₇-C₁₆arylalkyl or (C₄-C₁₀heteroaryl)methyl are independently substituted with 0-3 C₁-C₆alkyl, C₁-C₆alkoxy, halo, CF₃, OCF₃, hydroxy, C₁-C₅alkylC(O)O—, nitro, —CN, C₂-C₆alkenyl, or C₂-C₆alkynyl groups; [symbol]
represents an S-isomer, R-isomer or racemate;
the process comprising reacting HN(R³)(R⁴) with a compound of formula IA:

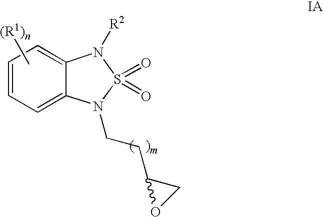

wherein the compound of formula I is formed.

Another aspect of the invention provides a process for the preparation of a compound of formula IA:

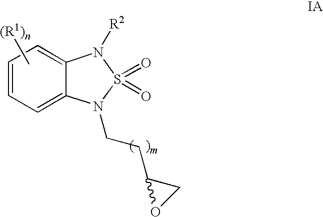

or a tautomer or salt thereof;
wherein:
m is an integer from 1 to 3;
n is an integer from 0 to 4;
R¹ is, independently at each occurrence, C₁-C₆alkyl, C₁-C₆alkoxy, halo, CF₃, OCF₃, hydroxy, C₁-C₅alkylC(O)O—, nitro, —CN, C₂-C₆alkenyl, C₂-C₆alkynyl, C₆-C₁₀aryl, C₄-C₁₀heteroaryl, C₁-C₆alkylS(O)—, C₁-C₆alkylS(O)₂—, C₁-C₆alkylS(O)₂NH—, C₁-C₆alkylS(O)₂N(C₁-C₆alkyl)-, C₆-C₁₀arylS(O)₂NH—, C₆-C₁₀arylS(O)₂N(C₁-C₆alkyl)-, C₁-C₅alkylC(O)NH—, C₁-C₅alkylC(O)N(C₁-C₆alkyl)-, or C₆-C₁₀arylC(O)NH—, C₆-C₁₀arylC(O)N(C₁-C₆alkyl)-; wherein each C₆-C₁₀aryl or C₄-C₁₀heteroaryl is independently substituted with 0-3 C₁-C₆alkyl, C₁-C₆alkoxy, halo, CF₃, OCF₃, hydroxy, C₁-C₅alkylC(O)O—, nitro, —CN, C₂-C₆alkenyl, or C₂-C₆alkynyl groups; and each C₆-C₁₀arylS(O)₂NH—, C₆-C₁₀arylS(O)₂N(C₁-C₆alkyl)-, or C₆-C₁₀arylC(O)NH—, C₆-C₁₀arylC(O)N(C₁-C₆alkyl)- is independently substituted with 0-3 C₁-C₆alkyl, C₁-C₆alkoxy, halo, CF₃, OCF₃, hydroxy, C₁-C₅alkylC(O)O—, nitro, —CN, C₂-C₆alkenyl, C₂-C₆alkynyl, C₁-C₆alkylS(O)—, C₁-C₆alkylS(O)₂—, C₁-C₆alkylS(O)₂NH—, C₁-C₆alkylS(O)₂N(C₁-C₆alkyl)-, C₁-C₅alkylC(O)NH—, or C₁-C₅alkylC(O)N(C₁-C₆alkyl)- groups;
R² is C₆-C₁₀aryl or C₄-C₁₀heteroaryl substituted with 0-5 C₁-C₆alkyl, C₁-C₆alkoxy, halo, CF₃, OCF₃, hydroxy, C₁-C₅alkylC(O)O—, nitro, —CN, C₂-C₆alkenyl, C₂-C₆alkynyl, C₁-C₆alkylS(O)—, C₁-C₆alkylS(O)₂—, C₁-C₆alkylS(O)₂NH—, C₁-C₆alkylS(O)₂N(C₁-C₆alkyl)-, C₆-C₁₀arylS(O)₂NH—, C₆-C₁₀arylS(O)₂N(C₁-C₆alkyl)-, C₁-C₅alkylC(O)NH—, C₁-C₅alkylC(O)N(C₁-C₆alkyl)-, C₆-C₁₀arylC(O)NH—, C₆-C₁₀arylC(O)N(C₁-C₆alkyl)-, or C₆-C₁₀aryl or C₄-C₁₀heteroaryl optionally substituted with C₁-C₆alkyl, C₁-C₆alkoxy, halo, CF₃, OCF₃, hydroxy, C₁-C₅alkylC(O)O—, nitro, —CN, C₂-C₆alkenyl, or C₂-C₆alkynyl; and [symbol]
represents an S-isomer, R-isomer or racemate;
the process comprising:
reacting a compound of formula IB:

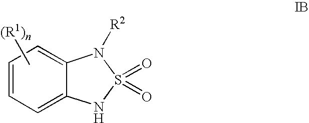

with a compound of formula IC:

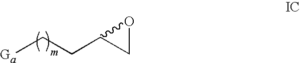

wherein Ga is an activating group.

Another aspect of the invention provides a process for the preparation of a compound of formula IB:

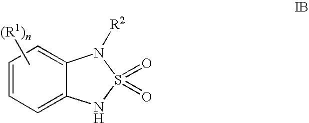

or a tautomer or salt thereof;
wherein:
n is an integer from 0 to 4;
R¹ is, independently at each occurrence, C₁-C₆alkyl, C₁-C₆alkoxy, halo, CF₃, OCF₃, hydroxy, C₁-C₅alkylC(O)O—, nitro, —CN, C₂-C₆alkenyl, C₂-C₆alkynyl, C₆-C₁₀aryl, C₄-C₁₀heteroaryl, C₁-C₆alkylS(O)—, C₁-C₆alkylS(O)₂—, C₁-C₆alkylS(O)₂NH—, C₁-C₆alkylS(O)₂N(C₁-C₆alkyl)-, C₆-C₁₀arylS(O)₂NH—, C₆-C₁₀arylS(O)₂N(C₁-C₆alkyl)-, C₁-C₅alkylC(O)NH—, C₁-C₅alkylC(O)N(C₁-C₆alkyl)-, or C₆-C₁₀arylC(O)NH—, C₆-C₁₀arylC(O)N(C₁-C₆alkyl)-; wherein each C₆-C₁₀aryl or C₄-C₁₀heteroaryl is independently substituted with 0-3 C₁-C₆alkyl, C₁-C₆alkoxy, halo, CF₃, OCF₃, hydroxy, C₁-C₅alkylC(O)O—, nitro, —CN, C₂-C₆alkenyl, or C₂-C₆alkynyl groups; and each C₆-C₁₀arylS(O)₂NH—, C₆-C₁₀arylS(O)₂N(C₁-C₆alkyl)-, or C₆-C₁₀arylC(O)NH—, C₆-C₁₀arylC(O)N(C₁-C₆alkyl)- is independently substituted with 0-3 C₁-C₆alkyl, C₁-C₆alkoxy, halo, CF₃, OCF₃, hydroxy, C₁-C₅alkylC(O)O—, nitro, —CN, C₂-C₆alkenyl, C₂-C₆alkynyl, C₁-C₆alkylS(O)—, C₁-C₆alkylS(O)₂—, C₁-C₆alkylS(O)₂NH—, C₁-C₆alkylS(O)₂N(C₁-C₆alkyl)-, C₁-C₅alkylC(O)NH—, or C₁-C₅alkylC(O)N(C₁-C₆alkyl)- groups; and
R² is C₆-C₁₀aryl or C₄-C₁₀heteroaryl substituted with 0-5 C₁-C₆alkyl, C₁-C₆alkoxy, halo, CF₃, OCF₃, hydroxy, C₁-C₅alkylC(O)O—, nitro, —CN, C₂-C₆alkenyl, C₂-C₆alkynyl, C₁-C₆alkylS(O)—, C₁-C₆alkylS(O)₂—, C₁-C₆alkylS(O)₂NH—, C₁-C₆alkylS(O)₂N(C₁-C₆alkyl)-, C₆-C₁₀arylS(O)₂NH—, C₆-C₁₀arylS(O)₂N(C₁-C₆alkyl)-, C₁-C₅alkylC(O)NH—, C₁-C₅alkylC(O)N(C₁-C₆alkyl)-, C₆-C₁₀arylC(O)NH—, C₆-C₁₀arylC(O)N(C₁-C₆alkyl)-, or C₆-C₁₀aryl or C₄-C₁₀heteroaryl optionally substituted with C₁-C₆alkyl, C₁-C₆alkoxy, halo, CF₃, OCF₃, hydroxy, C₁-C₅alkylC(O)O—, nitro, —CN, C₂-C₆alkenyl, or C₂-C₆alkynyl;
the process comprising:
protecting a compound of formula IF:

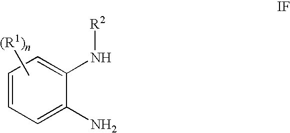

to form a compound of formula IG:

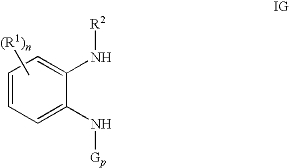

reacting SO(Ga)₂ with the compound of formula IG to form a compound of formula IH:

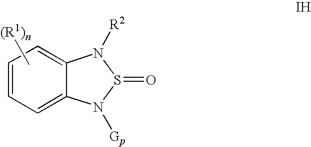

oxidizing the compound of formula IH to form a compound of formula IJ:

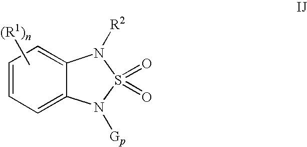

and deprotecting the compound of formula IJ to form the compound of formula IB.

Another aspect of the invention provides a compound comprising a compound of formula IA, IB, IC, ID, IE, IF, IG, IH, IJ, IK, or IL.

Another aspect of the invention provides a composition comprising:

(a) one or more of the compounds of formula IA, IB, IC, ID, IE, IF, IG, IH, IJ, IK, or IL; and
(b) one or more of: a base, an acid, a solvent, a hydrogenating agent, a reducing agent, an oxidizing agent, or a catalyst.

Other objects, features and advantages of the present invention will become apparent from the following detailed description. It should be understood, however, that the detailed description and the specific examples, while indicating embodiments of the invention, are given by way of illustration only, since various changes and modifications within the spirit and scope of the invention will become apparent to those skilled in the art from this detailed description.

### DETAILED DESCRIPTION

The following definitions are provided for the full understanding of terms and abbreviations used in this specification.

As used herein and in the appended claims, the singular forms “a,” “an,” and “the” include the plural reference unless the context clearly indicates otherwise. Thus, for example, “a compound” is a reference to one or more compounds and equivalents thereof known to those skilled in the art, “a catalyst” refers to one or more catalysts and equivalents thereof known to those skilled in the art, and so forth.

The abbreviations in the specification correspond to units of measure, techniques, properties, or compounds as follows: “min” means minutes, “h” means hour(s), “μL” means microliter(s), “mL” means milliliter(s), “mM” means millimolar, “M” means molar, “mmole” means millimole(s), “cm” means centimeters, “SEM” means standard error of the mean and “IU” means International Units. “° C.” refers to temperature in degree Celsius and “ED₅₀ value” means dose which results in 50% alleviation of the observed condition or effect (50% mean maximum endpoint).

The terms “component,” “composition,” “composition of compounds,” “compound,” “drug,” or “pharmacologically active agent” or “active agent” or “medicament” are used interchangeably herein to refer to a compound or compounds or composition of matter which, when administered to a subject (human or animal) induces a desired pharmacological and/or physiologic effect by local and/or systemic action.

The term “modulation” refers to the capacity to either enhance or inhibit a functional property of a biological activity or process; for example, receptor binding or signaling activity. Such enhancement or inhibition may be contingent on the occurrence of a specific event, such as activation of a signal transduction pathway and/or may be manifest only in particular cell types. The modulator is intended to comprise any compound; e.g., antibody, small molecule, peptide, oligopeptide, polypeptide, or protein, and is preferably small molecule, or peptide.

As used herein, the term “inhibitor” refers to any agent that inhibits, suppresses, represses, or decreases a specific activity, such as norepinephrine reuptake activity. The term “inhibitor” is intended to comprise any compound; e.g., antibody, small molecule, peptide, oligopeptide, polypeptide, or protein (preferably small molecule or peptide) that exhibits a partial, complete, competitive and/or inhibitory effect on mammalian (preferably the human) norepinephrine reuptake or both serotonin reuptake and the norepinephrine reuptake, thus diminishing or blocking (preferably diminishing) some or all of the biological effects of endogenous norepinephrine reuptake or of both serotonin reuptake and the norepinephrine reuptake.

Within the present invention, the compounds may be prepared in the form of salts and pharmaceutically acceptable salts. As used herein, the term “pharmaceutically acceptable salts” refers to salts prepared from pharmaceutically acceptable non-toxic acids, including inorganic salts and organic salts. Suitable non-organic salts include inorganic and organic acids such as acetic, benzenesulfonic, benzoic, camphorsulfonic, citric, ethenesulfonic, fumaric, gluconic, glutamic, hydrobromic, hydrochloric, isethionic, lactic, malic, maleic, mandelic, methanesulfonic, mucic, nitric, pamoic, pantothenic, phosphoric, succinic, sulfuric, tartaric acid, p-toluenesulfonic and the like. Particularly preferred are hydrochloric, hydrobromic, phosphoric, and sulfuric acids, and most preferred is the hydrochloride salt. In the preparation of intermediates, any compatible salt can be used, toxic or non-toxic, for example Bu₄N⁺ salts.

“Administering,” as used herein, means either directly administering a compound or composition of the present invention, or administering a prodrug, derivative or analog which will form an equivalent amount of the active compound or substance within the body.

The term “subject” or “patient” refers to an animal including the human species that is treatable with the compounds, compositions, and/or methods of the present invention. The term “subject” or “subjects” is intended to refer to both the male and female gender unless one gender is specifically indicated. Accordingly, the term “patient” comprises any mammal, which may benefit from treatment or prevention of a disease or disorder, such as a human, especially if the mammal is female, either in the pre-menopausal, peri-menopausal, or post-menopausal period. Furthermore, the term patient includes female animals including humans and, among humans, not only women of advanced age who have passed through menopause but also women who have undergone hysterectomy or for some other reason have suppressed estrogen production, such as those who have undergone long-term administration of corticosteroids, suffer from Cushing's syndrome or have gonadal dysgenesis. However, the term “patient” is not intended to be limited to a woman.

“Alkyl,” as used herein, refers to an optionally substituted, saturated straight, branched, or cyclic hydrocarbon having from about 1 to about 20 carbon atoms (and all combinations and subcombinations of ranges and specific numbers of carbon atoms therein), with from about 1 to about 8 carbon atoms or 1 to 6 carbon atoms (C₁-C₆) being preferred, and with from about 1 to about 4 carbon atoms, herein referred to as “lower alkyl”, being more preferred. Alkyl groups include, but are not limited to, methyl, ethyl, n-propyl, isopropyl, n-butyl, isobutyl, t-butyl, n-pentyl, cyclopentyl, cyclopropyl, isopentyl, neopentyl, n-hexyl, isohexyl, cyclohexyl, cyclooctyl, adamantyl, 3-methylpentyl, 2,2-dimethylbutyl, and 2,3-dimethylbutyl. A branched alkyl group has at least 3 carbon atoms (e.g., an isopropyl group), and in various embodiments, has up to 6 carbon atoms, i.e., a branched lower alkyl group. Examples of branched lower alkyl groups include, but are not limited to:

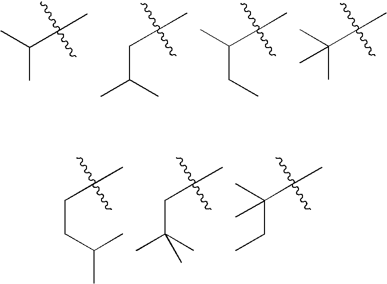

isopropyl, isobutyl, sec-butyl, tert-butyl, isopentyl, neopentyl, and tert-pentyl.

“Alkenyl,” as used herein, refers to an alkyl group of at least two carbon atoms having one or more double bonds, wherein alkyl is as defined herein. Preferred alkenyl groups have from 2 to 6 carbon atoms (C₂-C₆). Alkenyl groups can be optionally substituted.

“Alkynyl,” as used herein, refers to an alkyl group of at least two carbon atoms having one or more triple bonds, wherein alkyl is as defined herein. Preferred alkynyl groups have from 2 to 6 carbon atoms (C₂-C₆). Alkynyl groups can be optionally substituted.

“Alkylenyl”, “alkenylenyl”, “alkynylenyl”, and “arylenyl” refer to the subsets of alkyl, alkenyl, alkynyl and aryl groups, respectively, as defined herein, including the same residues as alkyl, alkenyl, alkynyl, and aryl but having two points of attachment within a chemical structure. Examples of C₁-C₆alkylenyl include methylenyl (—CH₂—), ethylenyl (—CH₂CH₂—), propylenyl (—CH₂CH₂CH₂—), and dimethylpropylenyl (—CH₂C(CH₃)₂CH₂—). Likewise, examples of C₂-C₆alkenylenyl include ethenylenyl (—CH═CH— and propenylenyl (—CH═CH—CH₂—). Examples of C₂-C₆alkynylenyl include ethynylenyl (—C≡C—) and propynylenyl (—C≡C—CH₂—). Examples of arylenyl groups include phenylenyl;

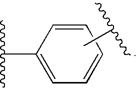

Preferably, arylenyl groups contain 6 carbon atoms (C₆).

“Halo,” as used herein, refers to chloro, bromo, fluoro, and iodo.

“Aryl” as used herein, refers to an optionally substituted, mono-, di-, tri-, or other multicyclic aromatic ring system having from about 5 to about 50 carbon atoms (and all combinations and subcombinations of ranges and specific numbers of carbon atoms therein), with from about 6 to about 10 carbons (C₆-C₁₀) being preferred. Non-limiting examples include, for example, phenyl, naphthyl, anthracenyl, and phenanthrenyl.

“Heteroaryl,” as used herein, refers to an optionally substituted, mono-, di-, tri-, or other multicyclic aromatic ring system that includes at least one, and preferably from 1 to about 4 heteroatom ring members selected from sulfur, oxygen and nitrogen. Heteroaryl groups can have, for example, from about 3 to about 50 carbon atoms (and all combinations and subcombinations of ranges and specific numbers of carbon atoms therein), with from about 4 to about 10 carbons being preferred. Non-limiting examples of C₄-C₁₀heteroaryl groups include, for example, pyrrolyl, furyl, pyridyl, 1,2,4-thiadiazolyl, pyrimidyl, thienyl, isothiazolyl, imidazolyl, tetrazolyl, pyrazinyl, pyrimidyl, quinolyl, isoquinolyl, thiophenyl, benzothienyl, isobenzofuryl, pyrazolyl, indolyl, purinyl, carbazolyl, benzimidazolyl, and isoxazolyl.

“Heterocyclic ring,” as used herein, refers to a stable 4- to 12-membered monocyclic or bicyclic or 7- to 10-membered bicyclic heterocyclic ring that is saturated, partially unsaturated or unsaturated (aromatic), and which contains carbon atoms and from 1 to 4 heteroatoms independently selected from the group consisting of N, O and S and including any bicyclic group in which any of the above defined heterocyclic rings is fused to a benzene ring. The nitrogen and sulfur heteroatoms may optionally be oxidized. The heterocyclic ring may be attached to its pendant group at any heteroatom or carbon atom that results in a stable structure. The heterocyclic rings described herein may be substituted on carbon or on a nitrogen atom if the resulting compound is stable. If specifically noted, a nitrogen atom in the heterocycle may optionally be quaternized. It is preferred that when the total number of S and O atoms in the heterocycle exceeds one, then these heteroatoms are not adjacent to one another. It is preferred that the total number of S and O atoms in the heterocycle is not more than two. Examples of heterocycles include, but are not limited to, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolyl, benzthiazolyl, benzotriazolyl, benzotetrazolyl, benzisoxazolyl, benzisothiazolyl, benzimidazalonyl, carbazolyl, 4H-carbazolyl, α-, β-, or γ-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylpyrimidinyl, phenanthridinyl, phenanthrolinyl, phenoxazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl. Preferred heterocycles include, but are not limited to, pyridinyl, furanyl, thienyl, pyrrolyl, pyrazolyl, imidazolyl, indolyl, benzimidazolyl, 1H-indazolyl, oxazolidinyl, benzotriazolyl, benzisoxazolyl, oxindolyl, benzoxazolinyl, or isatinyl. Also included are fused ring and spiro compounds containing, for example, the above heterocycles.

“Alkoxy,” as used herein, refers to the group R—O— where R is an alkyl group, as defined herein. Preferred alkoxy groups have from 1 to 6 carbon atoms (C₁-C₆).

“Arylalkyl,” as used herein, refers to the group R′—R— where R′ is a C₆-C₁₀aryl group, as defined herein, and R is a C₁-C₆alkyl group, as defined herein. Preferred arylalkyl groups have from 7 to 16 carbon atoms (C₇-C₁₆).

“Heteroarylalkyl,” as used herein, refers to the group R″—R— where R″ is a C₄-C₁₀heteroaryl group, as defined herein, and R is a C₁-C₆alkyl group, as defined herein.

“Heteroarylmethyl,” as used herein, refers to the group R″—CH₂— where R″ is a C₄-C₁₀heteroaryl group, as defined herein.

“Alkanoyloxy,” as used herein, refers to the group R—C(═O)—O— where R is a C₁-C₆alkyl group, as defined herein, of 1 to 5 carbon atoms (C₁-C₅).

“Alkylsulfoxide,” as used herein, refers to as used herein, refers to —S(═O)—R′, where R′ is C₁-C₆alkyl, as defined herein. Preferred alkysulfoxide groups have from 1 to 6 carbon atoms (C₁-C₆).

“Arylsulfoxide,” as used herein, refers to as used herein, refers to —S(═O)—R′, where R′ is C₆-C₁₀aryl, as defined herein. Preferred arylsulfoxide groups have from 6 to 10 carbon atoms (C₆-C₁₀).

“Alkylsulfone,” as used herein, refers to —S(═O)₂—R, where R is C₁-C₆alkyl, as defined herein. Preferred alkylsulfone groups have from 1 to 6 carbon atoms (C₁-C₆).

“Arylsulfone,” as used herein, refers to —S(═O)₂—R′, where R′ is C₆-C₁₀aryl, as defined herein. Preferred arylsulfone groups have from 6 to 10 carbon atoms (C₆-C₁₀).

“Alkylsulfonamide,” as used herein, refers to —NR—S(═O)₂—R, where each R is independently, C₁-C₆alkyl, as defined above, or the NR part may also be NH. Preferred alkylsulfonamide groups have from 1 to 6 carbon atoms (C₁-C₆).

“Arylsulfonamide,” as used herein, refers to —NR—S(═O)₂—R′, where R is H or C₁-C₆alkyl, as defined herein, and R′ is C₆-C₁₀aryl, as defined herein. Preferred arylsulfonamide groups have from 6 to 10 carbon atoms (C₆-C₁₀).

“Heteroarylsulfonamide,” as used herein, refers to —NR—S(═O)₂—R″, where R is H or C₁-C₆alkyl, as defined herein, and R″ is C₆-C₁₀aryl, as defined herein.

“Alkylamido,” as used herein, refers to —NR—C(═O)—R, where each R is independently, C₁-C₆alkyl, as defined above, or the NR part may also be NH. Preferred alkylamido groups have from 1 to 6 carbon atoms (C₁-C₆).

“Arylamido,” as used herein, refers to —NR—C(═O)—R″, where R is H or C₁-C₆alkyl, as defined herein, and R″ is C₆-C₁₀aryl, as defined herein. Preferred arylamido groups have from 6 to 10 carbon atoms (C₆-C₁₀).

“Phenylamido,” as used herein, refers to —NR—C(═O)-phenyl, where R is H or C₁-C₆alkyl, as defined above.

As used herein, the terms “optionally substituted” or “substituted or unsubstituted” are intended to refer to the optional replacement of up to four hydrogen atoms with up to four independently selected substituent groups as defined herein. Unless otherwise specified, suitable substituent groups independently include hydroxyl, nitro, amino, imino, cyano, halo, thio, sulfonyl, aminocarbonyl, carbonylamino, carbonyl, oxo, guanidine, carboxyl, formyl, C₁-C₆alkyl, perfluoroalkyl, alkyamino, dialkylamino, C₁-C₆alkoxy, alkoxyalkyl, alkylcarbonyl, arylcarbonyl, alkylthio, C₆-C₁₀aryl, C₄-C₁₀heteroaryl, a heterocyclic ring, cycloalkyl, hydroxyalkyl, carboxyalkyl, haloalkyl, C₂-C₆alkenyl, C₂-C₆alkynyl, C₇-C₁₆arylalkyl, aryloxy, heteroaryloxy, heteroarylalkyl, and the like. Substituent groups that have one or more available hydrogen atoms can in turn optionally bear further independently selected substituents, to a maximum of three levels of substitutions. For example, the term “optionally substituted C₁-C₆alkyl” is intended to mean an C₁-C₆alkyl group that can optionally have up to four of its hydrogen atoms replaced with substituent groups as defined above (i.e., a first level of substitution), wherein each of the substituent groups attached to the C₁-C₆alkyl group can optionally have up to four of its hydrogen atoms replaced by substituent groups as defined above (i.e., a second level of substitution), and each of the substituent groups of the second level of substitution can optionally have up to four of its hydrogen atoms replaced by substituent groups as defined above (i.e., a third level of substitution).

Unless indicated otherwise, the nomenclature of substituents that are not explicitly defined herein are arrived at by naming the terminal portion of the functionality followed by the adjacent functionality toward the point of attachment. For example, the substituent “arylalkoxycabonyl” refers to the group (C₆-C₁₀aryl)-(C₁-C₆alkyl)-O—C(O)—.

It is understood that the above definitions are not intended to include impermissible substitution patterns (e.g., methyl substituted with 5 fluoro groups). Such impermissible substitution patterns are well known to the skilled artisan.

At various places in the present specification, substituents of compounds are disclosed in groups or in ranges. It is specifically intended that the description include each and every individual subcombination of the members of such groups and ranges. For example, the term “C₁-C₆alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₈, C₃-C₄, C₄-C₆, C₄-C₅, and C₅-C₆ alkyl. By way of another example, the term “5-9 membered heteroaryl group” is specifically intended to individually disclose a heteroaryl group having 5, 6, 7, 8, 9, 5-9, 5-8, 5-7, 5-6, 6-9, 6-8, 6-7, 7-9, 7-8, and 8-9 ring atoms.

The term “protecting group” or “Gp” with respect to amine groups, hydroxyl groups and sulfhydryl groups refers to forms of these functionalities which are protected from undesirable reaction with a protecting group known to those skilled in the art, such as those set forth in Protective Groups in Organic Synthesis, Greene, T. W.; Wuts, P. G. M., John Wiley & Sons, New York, N.Y., (3rd Edition, 1999), the entire disclosure of which is herein incorporated by reference, which protecting groups can be added or removed using the procedures set forth therein. Examples of protected hydroxyl groups include, but are not limited to, silyl ethers such as those obtained by reaction of a hydroxyl group with a reagent such as, but not limited to, t-butyldimethyl-chlorosilane, trimethylchlorosilane, triisopropylchlorosilane, triethylchlorosilane; substituted methyl and ethyl ethers such as, but not limited to methoxymethyl ether, methythiomethyl ether, benzyloxymethyl ether, t-butoxymethyl ether, 2-methoxyethoxymethyl ether, tetrahydropyranyl ethers, 1-ethoxyethyl ether, allyl ether, benzyl ether; esters such as, but not limited to, benzoylformate, formate, acetate, trichloroacetate, and trifluoracetate. Examples of protected amine groups include, but are not limited to, amides such as, formamide, acetamide, trifluoroacetamide, and benzamide; carbamates; e.g. BOC; imides, such as phthalimide, Fmoc, Cbz, PMB, benzyl, and dithiosuccinimide; and others. Examples of protected or capped sulfhydryl groups include, but are not limited to, thioethers such as S-benzyl thioether, and S-4-picolyl thioether; substituted S-methyl derivatives such as hemithio, dithio and aminothio acetals; and others.

Reference to “activated” or “an activating group” or “Ga” as used herein indicates having an electrophilic moiety bound to a substituent, capable of being displaced by a nucleophile. Examples of preferred activating groups are halogens, such as F, Cl, Br or I; triflate; mesylate, or tosylate; esters; aldehydes; ketones; epoxides; and the like. An example of an activated group is acetylchloride, which is readily attacked by a nucleophile, such as piperidine group to form a N-acetylpiperidine functionality.

The term “deprotecting” refers to removal of a protecting group, such as removal of a benzyl or BOC group bound to an amine. Deprotecting may be preformed by heating and/or addition of reagents capable of removing protecting groups. In preferred embodiments, the deprotecting step involves addition of an acid, base, reducing agent, oxidizing agent, heat, or any combination thereof. One preferred method of removing BOC groups from amino groups is to add HCl in ethyl acetate. Many deprotecting reactions are well known in the art and are described in Protective Groups in Organic Synthesis, Greene, T. W., John Wiley & Sons, New York, N.Y., (1st Edition, 1981), the entire disclosure of which is herein incorporated by reference.

One aspect of the present invention provides a process comprising reacting HN(R³)(R⁴) with a compound of formula IA:

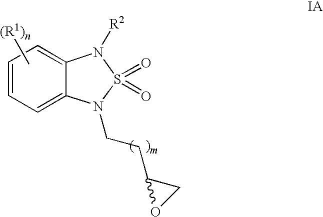

to give a compound of formula I:

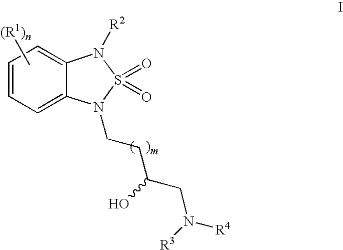

or a tautomer or pharmaceutically acceptable salt thereof;
wherein:
m is an integer from 1 to 3;
n is an integer from 0 to 4;
R¹ is, independently at each occurrence, C₁-C₆alkyl, C₁-C₆alkoxy, halo, CF₃, OCF₃, hydroxy, C₁-C₅alkylC(O)O—, nitro, —CN, C₂-C₆alkenyl, C₂-C₆alkynyl, C₆-C₁₀aryl, C₄-C₁₀heteroaryl, C₁-C₆alkylS(O)—, C₁-C₆alkylS(O)₂—, C₁-C₆alkylS(O)₂NH—, C₁-C₆alkylS(O)₂N(C₁-C₆alkyl)-, C₆-C₁₀arylS(O)₂NH—, C₆-C₁₀arylS(O)₂N(C₁-C₆alkyl)- C₁-C₅alkylC(O)NH—, C₁-C₅alkylC(O)N(C₁-C₆alkyl)-, or C₆-C₁₀arylC(O)NH—, C₆-C₁₀arylC(O)N(C₁-C₆alkyl)-; wherein each C₆-C₁₀aryl or C₄-C₁₀heteroaryl is independently substituted with 0-3 C₁-C₆alkyl, C₁-C₆alkoxy, halo, CF₃, OCF₃, hydroxy, C₁-C₅alkylC(O)O—, nitro, —CN, C₂-C₆alkenyl, or C₂-C₆alkynyl groups; and each C₆-C₁₀arylS(O)₂NH—, C₆-C₁₀arylS(O)₂N(C₁-C₆alkyl)- or C₆-C₁₀arylC(O)NH—, C₆-C₁₀arylC(O)N(C₁-C₆alkyl)- is independently substituted with 0-3 C₁-C₆alkyl, C₁-C₆alkoxy, halo, CF₃, OCF₃, hydroxy, C₁-C₅alkylC(O)O—, nitro, —CN, C₂-C₆alkenyl, C₂-C₆alkynyl, C₁-C₆alkylS(O)—, C₁-C₆alkylS(O)₂—, C₁-C₆alkylS(O)₂NH—, C₁-C₆alkylS(O)₂N(C₁-C₆alkyl)-, C₁-C₅alkylC(O)NH—, or C₁-C₅alkylC(O)N(C₁-C₆alkyl)- groups;
R² is C₆-C₁₀aryl or C₄-C₁₀heteroaryl substituted with 0-5 C₁-C₆alkyl, C₁-C₆alkoxy, halo, CF₃, OCF₃, hydroxy, C₁-C₅alkylC(O)O—, nitro, —CN, C₂-C₆alkenyl, C₂-C₆alkynyl, C₁-C₆alkylS(O)—, C₁-C₆alkylS(O)₂—, C₁-C₆alkylS(O)₂NH—, C₁-C₆alkylS(O)₂N(C₁-C₆alkyl)-, C₆-C₁₀arylS(O)₂NH—, C₆-C₁₀arylS(O)₂N(C₁-C₆alkyl)-, C₁-C₅alkylC(O)NH—, C₁-C₅alkylC(O)N(C₁-C₆alkyl)-, C₆-C₁₀arylC(O)NH—, C₆-C₁₀arylC(O)N(C₁-C₆alkyl)-, or C₆-C₁₀aryl or C₄-C₁₀heteroaryl optionally substituted with C₁-C₆alkyl, C₁-C₆alkoxy, halo, CF₃, OCF₃, hydroxy, C₁-C₅alkylC(O)O—, nitro, —CN, C₂-C₆alkenyl, or C₂-C₆alkynyl;
R³ and R⁴ are, independently, H, C₁-C₆alkyl, C₇-C₁₆arylalkyl or (C₄-C₁₀heteroaryl)methyl, wherein each of C₇-C₁₆arylalkyl or (C₄-C₁₀heteroaryl)methyl are independently substituted with 0-3 C₁-C₆alkyl, C₁-C₆alkoxy, halo, CF₃, OCF₃, hydroxy, C₁-C₅alkylC(O)O—, nitro, —CN, C₂-C₆alkenyl, or C₂-C₆alkynyl groups; [symbol]
represents an S-isomer, R-isomer or racemate;
wherein the compound of formula I is formed.

In a more particular embodiment, R² is:

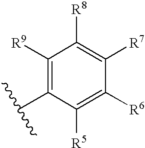

wherein,
each R⁵, R⁶, R⁷, R⁸ and R⁹ are independently selected from the group consisting of H, C₁-C₆alkyl, C₁-C₆alkoxy, halo, CF₃, OCF₃, hydroxy, C₁-C₅alkylC(O)O—, nitro, —CN, C₂-C₆alkenyl, C₂-C₆alkynyl, C₁-C₆alkylS(O)—, C₁-C₆alkylS(O)₂—, C₁-C₆alkylS(O)₂NH—, C₁-C₆alkylS(O)₂N(C₁-C₆alkyl)-, C₆-C₁₀arylS(O)₂NH—, C₆-C₁₀arylS(O)₂N(C₁-C₆alkyl)-, C₁-C₅alkylC(O)NH—, C₁-C₅alkylC(O)N(C₁-C₆alkyl)-, C₆-C₁₀arylC(O)NH—, C₆-C₁₀arylC(O)N(C₁-C₆alkyl)-, or C₆-C₁₀aryl or C₄-C₁₀heteroaryl optionally substituted with C₁-C₆alkyl, C₁-C₆alkoxy, halo, CF₃, OCF₃, hydroxy, C₁-C₅alkylC(O)O—, nitro, —CN, C₂-C₆alkenyl, or C₂-C₆alkynyl.

In a more particular embodiment, R⁹ is F. More particular still, R⁵, R⁶, R⁷ and R⁸ are H. In another embodiment, R⁵, R⁶, R⁷, R⁸ and R⁹ are H, halo, C₁-C₆alkyl or C₁-C₆alkoxy.

In a more particular embodiment, R³ is methyl. More particular still, R⁴ is H.

In a more particular embodiment, m is 1. More particular still, n is 0.

In a more particular embodiment, represents an S-isomer.

In a more particular embodiment:

R³ is methyl;

### R⁴ is H;

### R⁵, R⁶ R⁷ and R⁸ are H;

### R⁹ is F;

m is 1;
n is 0; and [symbol] represents an S-isomer.

In a more particular embodiment, the compound of formula I is:

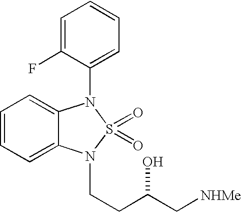

or pharmaceutically acceptable salt thereof.

In another embodiment, the compound of formula I is a hydrochloride or dihydrochloride salt and the hydrochloride or dihydrochloride salt is prepared by contacting the compound of formula I with anhydrous hydrochloric acid.

In another embodiment, the reacting step is performed in water and/or Me-THF.

Another aspect of the invention provides a process for the preparation of a compound of formula IA:

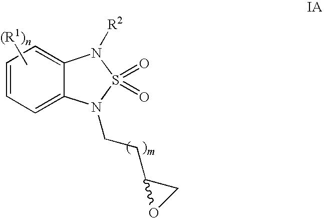

or a tautomer or salt thereof;
wherein:
m is an integer from 1 to 3;
n is an integer from 0 to 4;
R¹ is, independently at each occurrence, C₁-C₆alkyl, C₁-C₆alkoxy, halo, CF₃, OCF₃, hydroxy, C₁-C₅alkylC(O)O—, nitro, —CN, C₂-C₆alkenyl, C₂-C₆alkynyl, C₆-C₁₀aryl, C₄-C₁₀heteroaryl, C₁-C₆alkylS(O)—, C₁-C₆alkylS(O)₂—, C₁-C₆alkylS(O)₂NH—, C₁-C₆alkylS(O)₂N(C₁-C₆alkyl)-, C₆-C₁₀arylS(O)₂NH—, C₆-C₁₀arylS(O)₂N(C₁-C₆alkyl)- C₁-C₅alkylC(O)NH—, C₁-C₅alkylC(O)N(C₁-C₆alkyl)-, or C₆-C₁₀arylC(O)NH—, C₆-C₁₀arylC(O)N(C₁-C₆alkyl)-; wherein each C₆-C₁₀aryl or C₄-C₁₀heteroaryl is independently substituted with 0-3 C₁-C₆alkyl, C₁-C₆alkoxy, halo, CF₃, OCF₃, hydroxy, C₁-C₅alkylC(O)O—, nitro, —CN, C₂-C₆alkenyl, or C₂-C₆alkynyl groups; and each C₆-C₁₀arylS(O)₂NH—, C₆-C₁₀arylS(O)₂N(C₁-C₆alkyl)- or C₆-C₁₀arylC(O)NH—, C₆-C₁₀arylC(O)N(C₁-C₆alkyl)- is independently substituted with 0-3 C₁-C₆alkyl, C₁-C₆alkoxy, halo, CF₃, OCF₃, hydroxy, C₁-C₅alkylC(O)O—, nitro, —CN, C₂-C₆alkenyl, C₂-C₆alkynyl, C₁-C₆alkylS(O)—, C₁-C₆alkylS(O)₂—, C₁-C₆alkylS(O)₂NH—, C₁-C₆alkylS(O)₂N(C₁-C₆alkyl)-, C₁-C₅alkylC(O)NH—, or C₁-C₅alkylC(O)N(C₁-C₆alkyl)- groups;
R² is C₆-C₁₀aryl or C₄-C₁₀heteroaryl substituted with 0-5 C₁-C₆alkyl, C₁-C₆alkoxy, halo, CF₃, OCF₃, hydroxy, C₁-C₅alkylC(O)O—, nitro, —CN, C₂-C₆alkenyl, C₂-C₆alkynyl, C₁-C₆alkylS(O)—, C₁-C₆alkylS(O)₂—, C₁-C₆alkylS(O)₂NH—, C₁-C₆alkylS(O)₂N(C₁-C₆alkyl)-, C₆-C₁₀arylS(O)₂NH—, C₆-C₁₀arylS(O)₂N(C₁-C₆alkyl)-, C₁-C₅alkylC(O)NH—, C₁-C₅alkylC(O)N(C₁-C₆alkyl)-, C₆-C₁₀arylC(O)NH—, C₆-C₁₀arylC(O)N(C₁-C₆alkyl)-, or C₆-C₁₀aryl or C₄-C₁₀heteroaryl optionally substituted with C₁-C₆alkyl, C₁-C₆alkoxy, halo, CF₃, OCF₃, hydroxy, C₁-C₅alkylC(O)O—, nitro, —CN, C₂-C₆alkenyl, or C₂-C₆alkynyl; and [symbol]
represents an S-isomer, R-isomer or racemate;
the process comprising:
reacting a compound of formula IB:

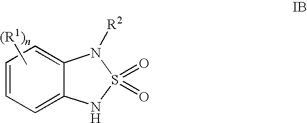

in the presence of a base with a compound of formula IC:

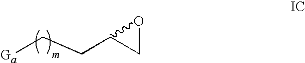

wherein, Ga is an activating group.

In another embodiment, the reacting step is performed in the presence of a base.

In another embodiment, the base is potassium carbonate (K₂CO₃).

In another embodiment, the reacting step is also performed in the presence of tetrabutylammonium iodide (TBAI) and Me-THF.

In another embodiment, Ga is halo, tosylate, mesylate, or triflate. More particularly, Ga is bromo (Br). Alternatively, Ga is tosylate.

In another embodiment, Ga is tosylate and the compound of formula IC is prepared by reacting tosyl chloride (TsCl) with a compound of formula ID in the presence of a base:

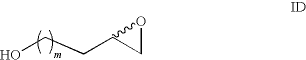

In another embodiment, the reacting step is performed in the presence of a base.

In another embodiment, the compound of formula ID is prepared by reacting a hydride and potassium phosphate (K₃PO₄) with a compound of formula IE:

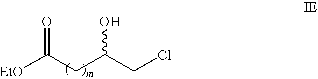

Another aspect of the invention provides the process wherein the compound of formula IB:

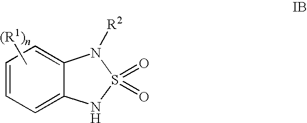

or a tautomer or salt thereof;
wherein:
n is an integer from 0 to 4;
R¹ is, independently at each occurrence, C₁-C₆alkyl, C₁-C₆alkoxy, halo, CF₃, OCF₃, hydroxy, C₁-C₅alkylC(O)O—, nitro, —CN, C₂-C₆alkenyl, C₂-C₆alkynyl, C₆-C₁₀aryl, C₄-C₁₀heteroaryl, C₁-C₆alkylS(O)—, C₁-C₆alkylS(O)₂—, C₁-C₆alkylS(O)₂NH—, C₁-C₆alkylS(O)₂N(C₁-C₆alkyl)-, C₆-C₁₀arylS(O)₂NH—, C₆-C₁₀arylS(O)₂N(C₁-C₆alkyl)- C₁-C₅alkylC(O)NH—, C₁-C₅alkylC(O)N(C₁-C₆alkyl)-, or C₆-C₁₀arylC(O)NH—, C₆-C₁₀arylC(O)N(C₁-C₆alkyl)-; wherein each C₆-C₁₀aryl or C₄-C₁₀heteroaryl is independently substituted with 0-3 C₁-C₆alkyl, C₁-C₆alkoxy, halo, CF₃, OCF₃, hydroxy, C₁-C₅alkylC(O)O—, nitro, —CN, C₂-C₆alkenyl, or C₂-C₆alkynyl groups; and each C₆-C₁₀arylS(O)₂NH—, C₆-C₁₀arylS(O)₂N(C₁-C₆alkyl)- or C₆-C₁₀arylC(O)NH—, C₆-C₁₀arylC(O)N(C₁-C₆alkyl)- is independently substituted with 0-3 C₁-C₆alkyl, C₁-C₆alkoxy, halo, CF₃, OCF₃, hydroxy, C₁-C₅alkylC(O)O—, nitro, —CN, C₂-C₆alkenyl, C₂-C₆alkynyl, C₁-C₆alkylS(O)—, C₁-C₆alkylS(O)₂—, C₁-C₆alkylS(O)₂NH—, C₁-C₆alkylS(O)₂N(C₁-C₆alkyl)-, C₁-C₅alkylC(O)NH—, or C₁-C₅alkylC(O)N(C₁-C₆alkyl)- groups; and
R² is C₆-C₁₀aryl or C₄-C₁₀heteroaryl substituted with 0-5 C₁-C₆alkyl, C₁-C₆alkoxy, halo, CF₃, OCF₃, hydroxy, C₁-C₅alkylC(O)O—, nitro, —CN, C₂-C₆alkenyl, C₂-C₆alkynyl, C₁-C₆alkylS(O)—, C₁-C₆alkylS(O)₂—, C₁-C₆alkylS(O)₂NH—, C₁-C₆alkylS(O)₂N(C₁-C₆alkyl)-, C₆-C₁₀arylS(O)₂NH—, C₆-C₁₀arylS(O)₂N(C₁-C₆alkyl)-, C₁-C₅alkylC(O)NH—, C₁-C₅alkylC(O)N(C₁-C₆alkyl)-, C₆-C₁₀arylC(O)NH—, C₆-C₁₀arylC(O)N(C₁-C₆alkyl)-, or C₆-C₁₀aryl or C₄-C₁₀heteroaryl optionally substituted with C₁-C₆alkyl, C₁-C₆alkoxy, halo, CF₃, OCF₃, hydroxy, C₁-C₅alkylC(O)O—, nitro, —CN, C₂-C₆alkenyl, or C₂-C₆alkynyl;
is prepared by a process comprising protecting a compound of formula IF:

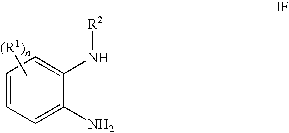

to form a compound of formula IG:

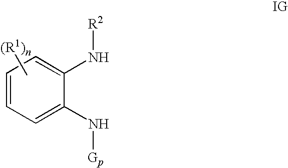

reacting SO(Ga)₂, wherein Ga is an activating group, with the compound of formula IG to form a compound of formula IH:

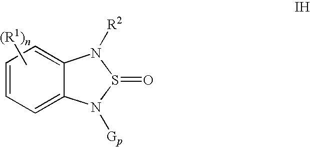

oxidizing the compound of formula IH to form a compound of formula IJ:

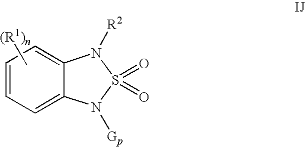

and deprotecting the compound of formula IJ to form the compound of formula IB.

In another embodiment, Ga is a halogen, such as F, Cl, Br or I; Ga is triflate; mesylate, or tosylate. Preferably, the reacting step is performed with thionyl chloride (SOCl₂).

In another embodiment, the Gp is selected from the group consisting of Boc, benzyl, acetyl, PMB, C₁-C₆alkyl, Fmoc, Cbz, trifluoroacetyl, tosyl and triphenylmethyl. More particularly, Gp is Boc.

In another embodiment, Gp is selected from the group consisting of Boc and the protecting step comprises reacting Boc anhydride (Boc₂O) with the compound of formula IF.

In another embodiment, the reacting step is performed in the presence of triethylamine (Et₃N).

In another embodiment, the oxidizing step is performed in the presence of ruthenium chloride (RuCl₃) and sodium periodate (NaIO₄). More particularly, the oxidizing step is performed in a biphasic toluene/water solution.

In another embodiment, the deprotecting step is performed in the presence of sodium methoxide (NaOMe) and toluene.

In another embodiment, the compound of formula IF is prepared by contacting R²—NH₂ with a compound of formula IK:

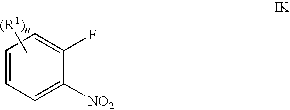

to form a compound of formula IL:

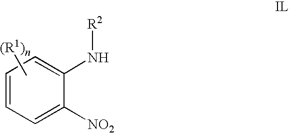

and hydrogenating the compound of formula IL to form the compound of formula IF.

In another embodiment, the contacting step is performed in the presence of potassium tertiary butoxide (t-BuOK).

In another embodiment, the hydrogenating step is performed in the presence of H₂ and palladium on carbon (Pd—C). More particularly, 0.5% palladium on carbon (Pd—C).

In another embodiment, the hydrogenating step is performed at about 0° C. or below.

In another embodiment, any of the process steps:

are performed at or above 30° C.;
are performed in a protic solvent, an aprotic solvent, a polar solvent, a nonpolar solvent, a protic polar solvent, an aprotic nonpolar solvent, or an aprotic polar solvent; or
include a purification step comprising at least one of: filtration, extraction, chromatography, trituration, or recrystallization.

Another aspect of the invention provides a compound comprising a compound of formula IA, IB, IC, ID, IE, IF, IG, IH, IJ, IK, or IL as described above.

Another aspect of the invention provides a composition comprising:

(c) one or more of the compounds of formula IA, IB, IC, ID, IE, IF, IG, IH, IJ, IK, or IL; and
(d) one or more of: a base, an acid, a solvent, a hydrogenating agent, a reducing agent, an oxidizing agent, or a catalyst.

Some of the compounds of the present invention may contain chiral centers and such compounds may exist in the form of stereoisomers (i.e. enantiomers or diastereomers). The present invention includes all such stereoisomers and any mixtures thereof including racemic mixtures. Racemic mixtures of the stereoisomers as well as the substantially pure stereoisomers are within the scope of the invention. The term “substantially pure,” as used herein, refers to at least about 90 mole %, more preferably at least about 95 mole %, and most preferably at least about 98 mole % of the desired stereoisomer is present relative to other possible stereoisomers. Preferred enantiomers may be isolated from racemic mixtures by any method known to those skilled in the art, including high performance liquid chromatography (HPLC) and the formation and crystallization of chiral salts or prepared by methods described herein. See, for example, Jacques, et al., Enantiomers, Racemates and Resolutions (Wiley Interscience, New York, 1981); Wilen, S. H., et al., Tetrahedron, 33:2725 (1977); Eliel, E. L. Stereochemistry of Carbon Compounds, (McGraw-Hill, NY, 1962); Wilen, S. H. Tables of Resolving Agents and Optical Resolutions, p. 268 (E. L. Eliel, Ed., University of Notre Dame Press, Notre Dame, Ind. 1972), the entire disclosures of which are herein incorporated by reference.

Further, the compounds of formula I may exist in unsolvated as well as in solvated forms with pharmaceutically acceptable solvents such as water, ethanol, and the like. In general, the solvated forms are considered equivalent to the unsolvated forms for the purpose of the present invention.

The compounds of formula I can be synthesized, for example, by the methods described below, or variations thereon as appreciated by the skilled artisan. All processes disclosed in association with the present invention are contemplated to be practiced on any scale, including milligram, gram, multigram, kilogram, multikilogram or commercial industrial scale.

As will be readily understood, functional groups present may contain protecting groups during the course of synthesis. Protecting groups are known per se as chemical functional groups that can be selectively appended to and removed from functionalities, such as hydroxyl groups and carboxyl groups. These groups are present in a chemical compound to render such functionality inert to chemical reaction conditions to which the compound is exposed. Any of a variety of protecting groups may be employed with the present invention. Protecting groups that may be employed in accordance with the present invention may be described in Greene, T. W. and Wuts, P. G. M., Protective Groups in Organic Synthesis 2d. Ed., Wiley & Sons, 1991, the entire disclosure of which is herein incorporated by reference.

Compounds of the present invention are suitably prepared in accordance with the following general description and specific examples. Variables used are as defined for formula I, unless otherwise noted. Reagents used in the preparation of the compounds of this invention can be either commercially obtained or can be prepared by standard procedures described in the literature. In accordance with this invention, compounds of formula I may be produced by the following reaction schemes.

Scheme 1 describes the synthesis of compounds of formula I through ten chemical transformations. This modified route is convergent and allows the introduction of the chiral side chain in the final step, thus minimizing the manipulation of chiral intermediates. Compounds synthesized by this route have 8-10% total yield.

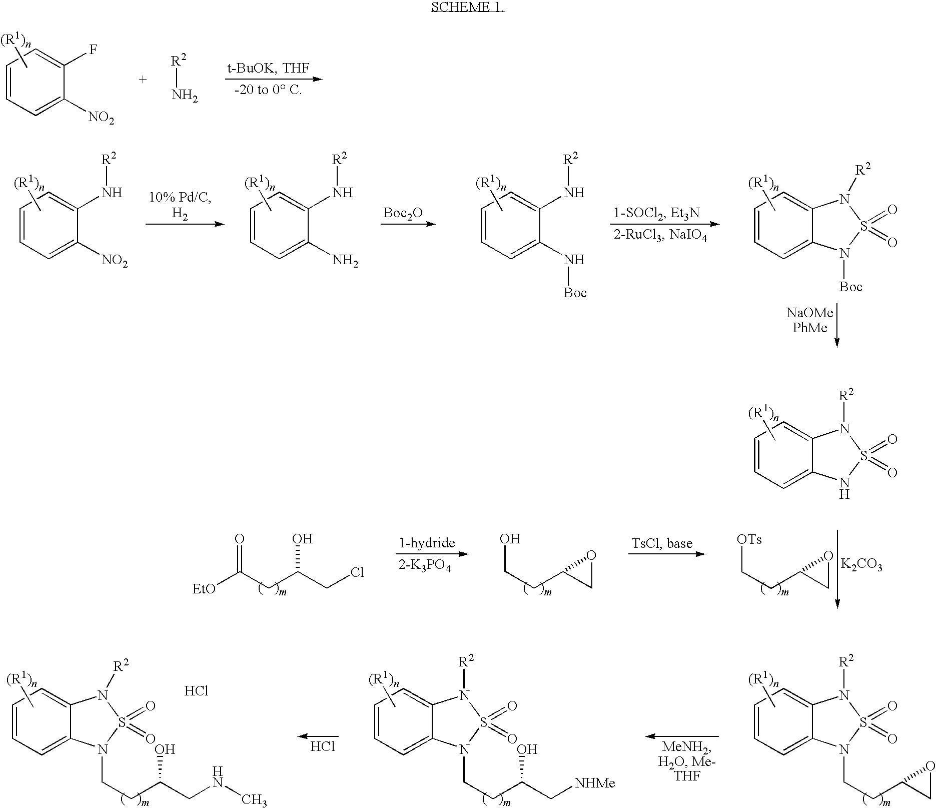

The synthesis begins by the reduction of the commercially available nitro aniline. The resulting dianiline is Boc protected in situ, which is then converted to the sulfoxide by treatment with thionyl chloride. Oxidation of sulfoxide affords the desired sulfone, which is subsequently deprotected under basic conditions. This is the core ring system onto which the side-chain is appended. The side-chain was synthesized via the epoxide alcohol. The tosyl epoxide is used in the alkylation step, which is further reacted with methylamine to afford the product as the free base. The HCl salt is obtained by treatment of the free base with anhydrous HCl.

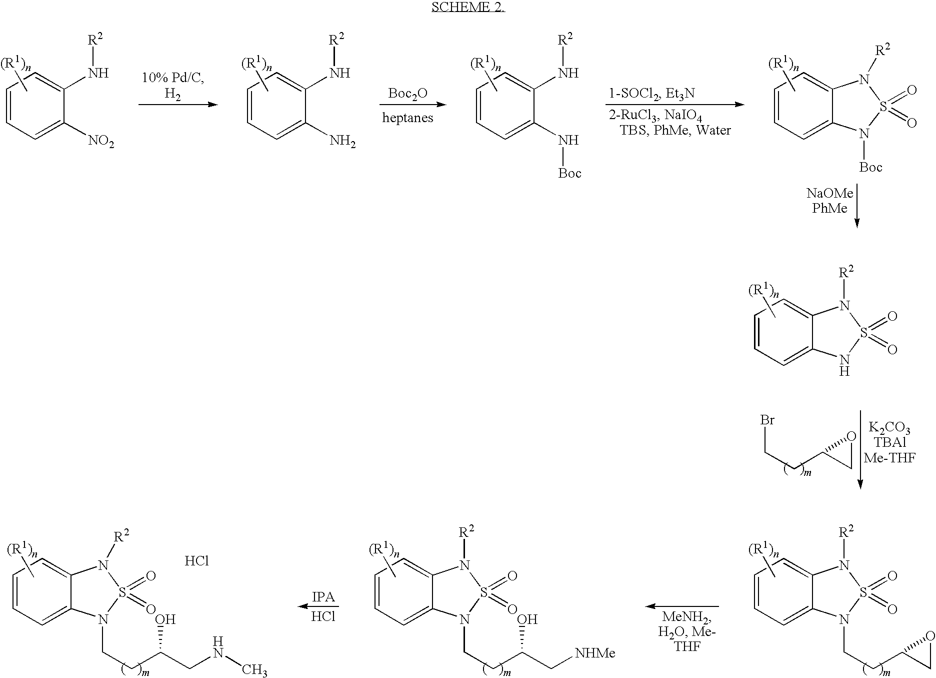

Scheme 2 contains 8 chemical transformations and the overall yield for compounds prepared by this route is expected to be approximately 25%.

The compounds of this invention contain chiral centers, providing for various stereoisomeric forms such as diastereomeric mixtures, enantiomeric mixtures as well as optical isomers. The individual optical isomers can be prepared directly through asymmetric and/or stereospecific synthesis or by conventional chiral separation of optical isomers from the enantiomeric mixture.

### EXAMPLES

### Example 1

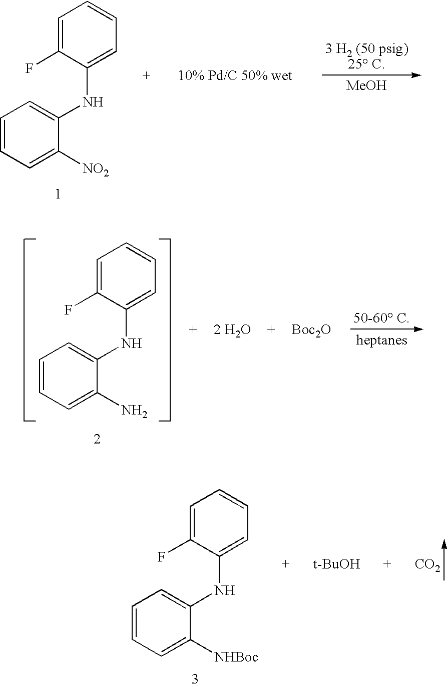

Hydrogenation of the nitro analogue (1) was performed in 2 wt % catalyst (Pd—C) at 40 psig H₂ with a moderate exotherm resulting in an average temperature rise of 15° C. in small scale Parr shaker run. After filtration of the Pd—C catalyst, di-t-butyl dicarbonate was added and the reaction stirred at room temperature until conversion was complete. The product was crystallized by solvent switching to heptane. The product was filtered and washed with heptane giving a brown/purple solid in 90% yield. Purity was ˜>98%. Alternatively, hydrogenation of 1 was performed in methanol at 50 psig with a maximum temperature of 42° C. and 97% yield after concentration to dryness. The protection of the amino group with di-tert-butyl dicarbonate was performed in dichloromethane and in heptanes. A one-pot reaction for the hydrogenation and protection was also performed. However, the fact that methanol reacts with di-t-butyl dicarbonate at a temperature of around 55° C. prevented the use of methanol for the one-pot process. A one-pot procedure in ethyl acetate was also developed with good preliminary results; however the gas evolution observed was problematic. Regardless the solvent used during the protection step, the amount of carbon dioxide generated during the process exceeded the pressure limit of hydrogenator.

In a preferred process, methanol and heptane were used and 0.5% of catalyst for the hydrogenation reaction (in contrast to previous use of 4%). The reduction proceeded in 2-6 h between 25-45° C. in MeOH with 0.5 wt % catalyst at 50 psi H₂. After filtration of the Pd—C catalyst, the amine (2) solution was concentrated followed by addition of heptanes. Di-t-butyl dicarbonate was added as a solution in heptanes to the amine (2) solution at 50-60° C. As the product 3 formed, precipitation was observed. After reaction completion, the product was filtered and washed with heptanes and dried under vacuum at 60° C. giving a purple/pink solid in 82% at 101.9% strength, 0.47% total impurities, 0.19% single largest impurity. Throughput was 7.8% (R) and 7.8% (W).

### Example 2

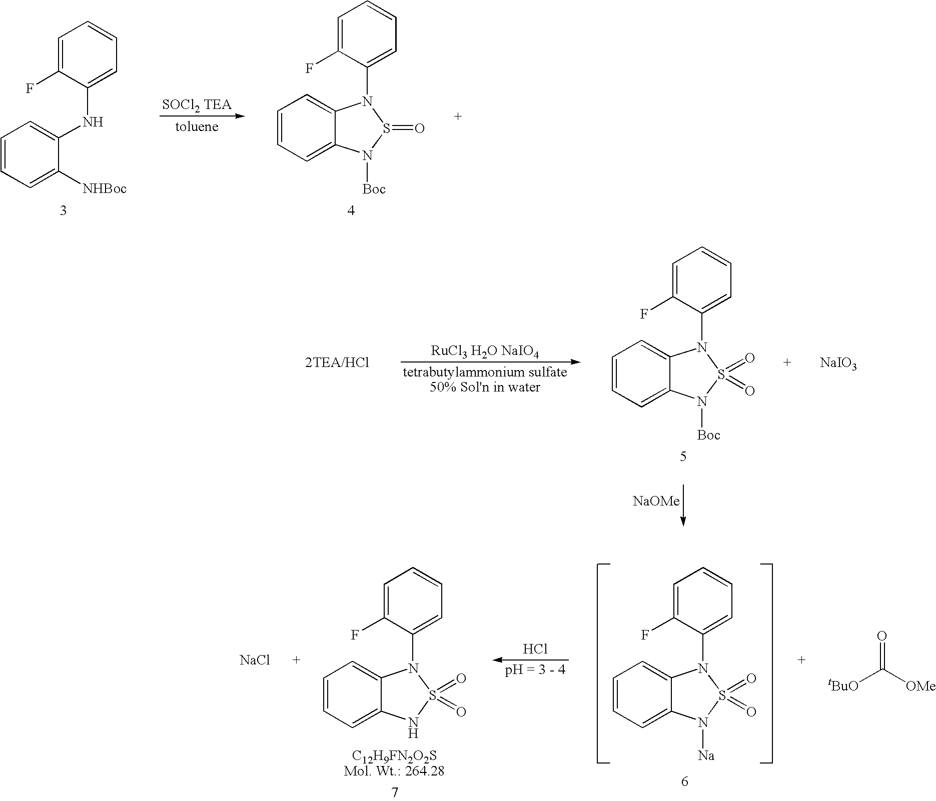

Prior procedures for preparing sulfamide (4) via direct sulfonylation of 2 have required harsh conditions (>150° C. reaction temperature), which can result in product that is degraded rapidly; low yield (i.e., in a range of 10 to 60%); and generation of tar and other impurities. Isolation and purification of the product was difficult.

In a preferred process, the amine (2) was first protected with a Boc group, then reacted with thionyl chloride at −10° C. to 0° C. to give sulfonamide. Toluene was used as the solvent (in place of CH₂Cl₂) as it can be used throughout multiple steps. The reaction at this step was conducted at −10 to 0° C. under addition control to avoid heat accumulation. After the addition of thionyl chloride, >90% of the starting material was converted to the product. The reaction mixture was then warmed to 20 to 35° C. so that the reaction went to completion because the starting material was only marginally soluble in toluene.

The original procedure for oxidation was carried out in acetonitrile. However, this procedure required multiple additions of catalyst and periodate to control exotherm, and the reaction took more than 12 h. Additionally, the reaction required removal of the by-product iodate solid from the reaction mixture and many impurities were generated from the reaction. Instead, by conducting the reaction in biphasic toluene/water in the presence of a phase transfer catalyst, the synthetic problems were alleviated. The exotherm was controlled by slowly adding aqueous periodate solution to the reaction. After the completion of the addition of periodate, >90% of the sulfinamide was converted to the product. Limiting the contact of the product and oxidant minimized the impurities. The by-product iodate stayed in the aqueous layer and was easy to remove.

Deprotection of Boc group from sulfamide (5) was originally conducted in acidic conditions such as TFA and HCl. It was found that the acidic conditions not only generated gas evolution, but also gave the product dark color that was difficult to remove. The inventors thus developed an improved procedure that used sodium methoxide that avoided gas evolution and color formation. After the completion of deprotection, the reaction was quenched with water. The product was extracted into the aqueous layer while the impurities remained in the organic layer. After acidification with HCl and extraction with toluene, the organic phase was concentrated under reduced pressure. The product was isolated as a beige solid, in a range of 70 to 90% yield and 97 to 99% of strength and purity. The reaction and work-up throughputs were about 2%. The major impurities found in the product were hydrolyzed sulfamide (MW 282) and des-fluoro sulfamide (MW 246), which may come from the hydrogenation step.

### Example 3

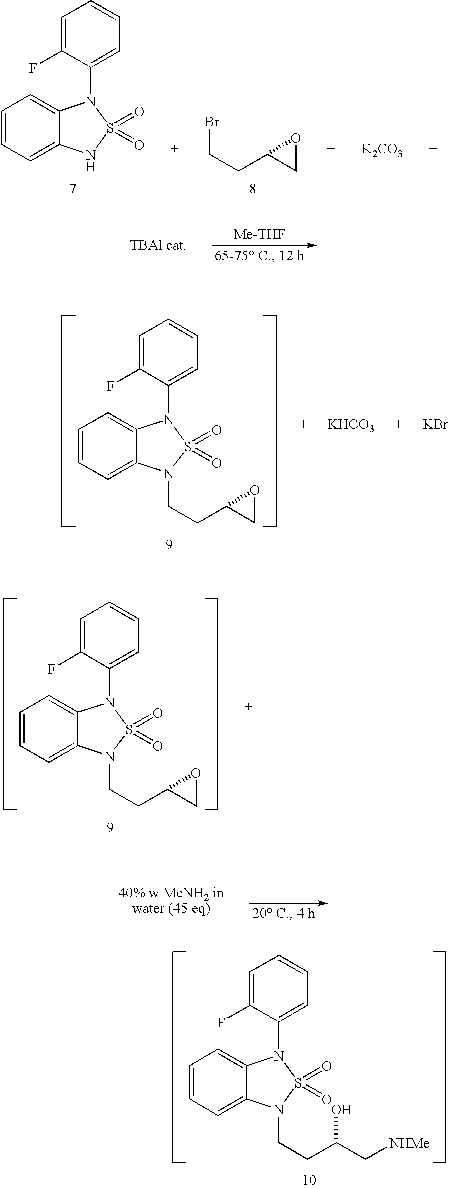

The following procedure was successful in the preparation of a 160 g batch, which used a tosyl epoxide side chain for the alkylation step. Improved quality of the bromo epoxide material (8) prompted a change to the bromo epoxide, which is commercially available from Suven at 98% purity. Iodide salt catalyst was necessary to ensure a good alkylation rate. Me-THF and elevated temperature were also used to obtain rapid and complete conversion. TBAI was preferred to other iodide salts because of a better purity profile during the reaction. The potassium carbonate was used in excess (3 equivalents) since it prevents the degradation of the sulfamide intermediate in Me-THF solution at 75° C. over time. In this reaction, the sulfamide (7) was added as a solution in Me-THF to a mixture of K₂CO₃, TBAI and bromo epoxide (8) in Me-THF at 65-75° C. After reaction completion (12 h), the solids were filtered and washed with Me-THF. The filtrate and washes were combined and concentrated to 8 volumes by vacuum distillation. This solution was only concentrated to 8 volumes because of safety concerns regarding the concentrated mixture. The alkylated product (9) is a gummy solid which was difficult to isolate, and therefore was telescoped as a solution in the next reaction.

In the epoxide-opening step, the main focus was to minimize the formation of dimeric impurities. The reaction was performed using a total of 26 volumes, including 45 equivalents of the methylamine solution, by adding compound 9 in 8 volumes of Me-THF to a mixture of methylamine in water with 2 volumes of THF. The THF helps to homogenize the mixture, thus preventing phase-split problems and ensuring faster reaction rate and reducing the dimer formation. The reaction was performed at room temperature to minimize the methylamine evaporation and the excess was removed by vacuum distillation after the reaction completion using a scrubber. The distillation was performed soon after reaction completion, since the free base can react with an additional molecule of methylamine by opening the sulfamide ring. The free base extraction was done using large volumes of toluene to ensure good recovery, since the product was water-soluble. The combined toluene extracts were distilled to a lower volume. The free base was not isolated as a solid and instead, was carried on to the next step as a toluene solution.

The expected yield for the alkylation step was around 90% based on strength of the solution. Typically, 15 to 20% impurities were present in the free base solution.

### Example 4

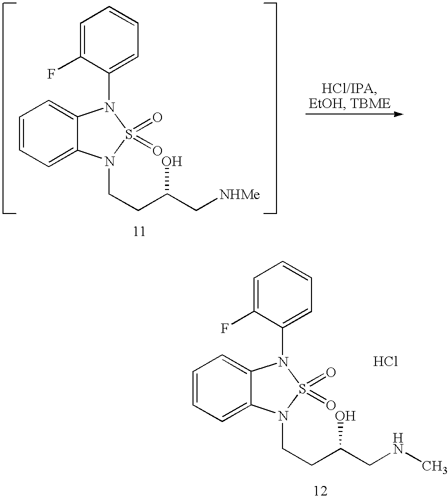

No isolation was done prior to the salt formation; thus, high levels of impurities were carried over in the last step having a major impact on the crystallization. Therefore, a seeding procedure is necessary to ensure constant results. It is important that the KF value be below 1% since the salt is highly soluble in water and, otherwise, “oiling out” or poor yield can be observed. Several purification methods were tried on the free base or the crude salt, with a significant improvement observed by use of acetonitrile.

By using acetonitrile during salt formation, the purity of the isolated salt was highly improved and no oiling issues (observed with ethanol) were detected. The HCl was added as a 2-propanol solution, since anhydrous reaction conditions were required. However, only one volume of IPA was added, since the product is highly soluble in a combination of IPA/acetonitrile. The pH of the mixture after HCl addition was between 2.5 and 3.5 for optimal results. At lower pH, degradation was observed. At high pH, there was no rejection of impurities. The ratio 1:2 of acetonitrile and TBME was optimized in order to control the purity profile obtained in the isolated solid (>99.5%).

When ranges are used herein for physical properties, such as molecular weight, or chemical properties, such as chemical formulae, all combinations and subcombinations of ranges specific embodiments therein are intended to be included.

The disclosures of each patent, patent application and publication cited or described in this document are hereby incorporated herein by reference, in its entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed is:

1. A compound formula IA: 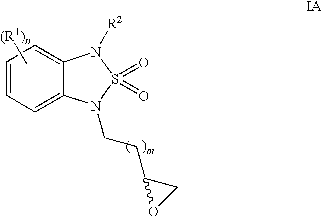
or a tautomer or salt thereof;
wherein:
m is an integer from 1 to 3;
n is an integer from 0 to 4;
R¹ is, independently at each occurrence, C₁-C₆alkyl, C₁-C₆alkoxy, halo, CF₃, OCF₃, hydroxy, C₁-C₅alkylC(O)O—, nitro, —CN, C₂-C₆alkenyl, C₂-C₆alkynyl, C₆-C₁₀aryl, C₄-C₁₀heteroaryl, C₁-C₆alkylS(O)—, C₁-C₆alkylS(O)₂—, C₁-C₆alkylS(O)₂NH—, C₁-C₆alkylS(O)₂N(C₁-C₆alkyl)-, C₆-C₁₀arylS(O)₂NH—, C₆-C₁₀arylS(O)₂N(C₁-C₆alkyl)- C₁-C₅alkylC(O)NH—, C₁-C₅alkylC(O)N(C₁-C₆alkyl)-, or C₆-C₁₀arylC(O)NH—, C₆-C₁₀arylC(O)N(C₁-C₆alkyl)-; wherein each C₆-C₁₀aryl or C₄-C₁₀heteroaryl is independently substituted with 0-3 C₁-C₆alkyl, C₁-C₆alkoxy, halo, CF₃, OCF₃, hydroxy, C₁-C₅alkylC(O)O—, nitro, —CN, C₂-C₆alkenyl, or C₂-C₆alkynyl groups; and each C₆-C₁₀arylS(O)₂NH—, C₆-C₁₀arylS(O)₂N(C₁-C₆alkyl)- or C₆-C₁₀arylC(O)NH—, C₆-C₁₀arylC(O)N(C₁-C₆alkyl)- is independently substituted with 0-3 C₁-C₆alkyl, C₁-C₆alkoxy, halo, CF₃, OCF₃, hydroxy, C₁-C₅alkylC(O)O—, nitro, —CN, C₂-C₆alkenyl, C₂-C₆alkynyl, C₁-C₆alkylS(O)—, C₁-C₆alkylS(O)₂—, C₁-C₆alkylS(O)₂NH—, C₁-C₆alkylS(O)₂N(C₁-C₆alkyl)-, C₁-C₅alkylC(O)NH—, or C₁-C₅alkylC(O)N(C₁-C₆alkyl)- groups;
R² is C₆-C₁₀aryl or C₄-C₁₀heteroaryl substituted with 0-5 C₁-C₆alkyl, C₁-C₆alkoxy, halo, CF₃, OCF₃, hydroxy, C₁-C₅alkylC(O)O—, nitro, —CN, C₂-C₆alkenyl, C₂-C₆alkynyl, C₁-C₆alkylS(O)—, C₁-C₆alkylS(O)₂—, C₁-C₆alkylS(O)₂NH—, C₁-C₆alkylS(O)₂N(C₁-C₆alkyl)-, C₆-C₁₀arylS(O)₂NH—, C₆-C₁₀arylS(O)₂N(C₁-C₆alkyl)-, C₁-C₅alkylC(O)NH—, C₁-C₅alkylC(O)N(C₁-C₆alkyl)-, C₆-C₁₀arylC(O)NH—, C₆-C₁₀arylC(O)N(C₁-C₆alkyl)-, or C₆-C₁₀aryl or C₄-C₁₀heteroaryl optionally substituted with C₁-C₆alkyl, C₁-C₆alkoxy, halo, CF₃, OCF₃, hydroxy, C₁-C₅alkylC(O)O—, nitro, —CN, C₂-C₆alkenyl, or C₂-C₆alkynyl; and [symbol]
represents an S-isomer, R-isomer or racemate.

2. A process comprising reacting HN(R³)(R⁴) with a compound of formula IA: 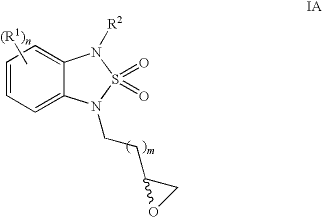
to give a compound of formula I: 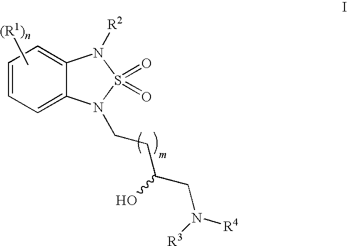
or a tautomer or pharmaceutically acceptable salt thereof;
wherein:
m is an integer from 1 to 3;
n is an integer from 0 to 4;
R¹ is, independently at each occurrence, C₁-C₆alkyl, C₁-C₆alkoxy, halo, CF₃, OCF₃, hydroxy, C₁-C₅alkylC(O)O—, nitro, —CN, C₂-C₆alkenyl, C₂-C₆alkynyl, C₆-C₁₀aryl, C₄-C₁₀heteroaryl, C₁-C₆alkylS(O)—, C₁-C₆alkylS(O)₂—, C₁-C₆alkylS(O)₂NH—, C₁-C₆alkylS(O)₂N(C₁-C₆alkyl)-, C₆-C₁₀arylS(O)₂NH—, C₆-C₁₀arylS(O)₂N(C₁-C₆alkyl)- C₁-C₅alkylC(O)NH—, C₁-C₅alkylC(O)N(C₁-C₆alkyl)-, or C₆-C₁₀arylC(O)NH—, C₆-C₁₀arylC(O)N(C₁-C₆alkyl)-; wherein each C₆-C₁₀aryl or C₄-C₁₀heteroaryl is independently substituted with 0-3 C₁-C₆alkyl, C₁-C₆alkoxy, halo, CF₃, OCF₃, hydroxy, C₁-C₅alkylC(O)O—, nitro, —CN, C₂-C₆alkenyl, or C₂-C₆alkynyl groups; and each C₆-C₁₀arylS(O)₂NH—, C₆-C₁₀arylS(O)₂N(C₁-C₆alkyl)- or C₆-C₁₀arylC(O)NH—, C₆-C₁₀arylC(O)N(C₁-C₆alkyl)- is independently substituted with 0-3 C₁-C₆alkyl, C₁-C₆alkoxy, halo, CF₃, OCF₃, hydroxy, C₁-C₅alkylC(O)O—, nitro, —CN, C₂-C₆alkenyl, C₂-C₆alkynyl, C₁-C₆alkylS(O)—, C₁-C₆alkylS(O)₂—, C₁-C₆alkylS(O)₂NH—, C₁-C₆alkylS(O)₂N(C₁-C₆alkyl)-, C₁-C₅alkylC(O)NH—, or C₁-C₅alkylC(O)N(C₁-C₆alkyl)- groups;
R² is C₆-C₁₀aryl or C₄-C₁₀heteroaryl substituted with 0-5 C₁-C₆alkyl, C₁-C₆alkoxy, halo, CF₃, OCF₃, hydroxy, C₁-C₅alkylC(O)O—, nitro, —CN, C₂-C₆alkenyl, C₂-C₆alkynyl, C₁-C₆alkylS(O)—, C₁-C₆alkylS(O)₂—, C₁-C₆alkylS(O)₂NH—, C₁-C₆alkylS(O)₂N(C₁-C₆alkyl)-, C₆-C₁₀arylS(O)₂NH—, C₆-C₁₀arylS(O)₂N(C₁-C₆alkyl)-, C₁-C₅alkylC(O)NH—, C₁-C₅alkylC(O)N(C₁-C₆alkyl)-, C₆-C₁₀arylC(O)NH—, C₆-C₁₀arylC(O)N(C₁-C₆alkyl)-, or C₆-C₁₀aryl or C₄-C₁₀heteroaryl optionally substituted with C₁-C₆alkyl, C₁-C₆alkoxy, halo, CF₃, OCF₃, hydroxy, C₁-C₅alkylC(O)O—, nitro, —CN, C₂-C₆alkenyl, or C₂-C₆alkynyl;
R³ and R⁴ are, independently, H, C₁-C₆alkyl, C₇-C₁₆arylalkyl or (C₄-C₁₀heteroaryl)methyl, wherein each of C₇-C₁₆arylalkyl or (C₄-C₁₀heteroaryl)methyl are independently substituted with 0-3 C₁-C₆alkyl, C₁-C₆alkoxy, halo, CF₃, OCF₃, hydroxy, C₁-C₅alkylC(O)O—, nitro, —CN, C₂-C₆alkenyl, or C₂-C₆alkynyl groups; [symbol]
represents an S-isomer, R-isomer or racemate;
wherein the compound of formula I is formed.

3. The process of claim 2, wherein R² is: 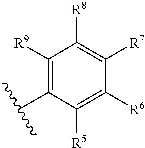
wherein,
each R⁵, R⁶, R⁷, R⁸ and R⁹ are independently selected from the group consisting of H, C₁-C₆alkyl, C₁-C₆alkoxy, halo, CF₃, OCF₃, hydroxy, C₁-C₅alkylC(O)O—, nitro, —CN, C₂-C₆alkenyl, C₂-C₆alkynyl, C₁-C₆alkylS(O)—, C₁-C₆alkylS(O)₂—, C₁-C₆alkylS(O)₂NH—, C₁-C₆alkylS(O)₂N(C₁-C₆alkyl)-, C₆-C₁₀arylS(O)₂NH—, C₆-C₁₀arylS(O)₂N(C₁-C₆alkyl)-, C₁-C₅alkylC(O)NH—, C₁-C₅alkylC(O)N(C₁-C₆alkyl)-, C₆-C₁₀arylC(O)NH—, C₆-C₁₀arylC(O)N(C₁-C₆alkyl)-, or C₆-C₁₀aryl or C₄-C₁₀heteroaryl optionally substituted with C₁-C₆alkyl, C₁-C₆alkoxy, halo, CF₃, OCF₃, hydroxy, C₁-C₅alkylC(O)O—, nitro, —CN, C₂-C₆alkenyl, or C₂-C₆alkynyl.

4. The process of claim 3, wherein R⁹ is F.

5. The process of claim 4, wherein R⁵, R⁶, R⁷ and R⁸ are H.

6. The process of claim 3, wherein R⁵, R⁶, R⁷, R⁸ and R⁹ are H, halo, C₁-C₆alkyl or C₁-C₆alkoxy.

7. The process of claim 2, wherein R³ is methyl.

8. The process of claim 2, wherein R⁴ is H.

9. The process of claim 2, wherein m is 1.

10. The process of claim 2, wherein n is 0.

11. The process of claim 2, wherein: [symbol] represents an S-isomer.

12. The process of claim 5, wherein the compound of formula I is: 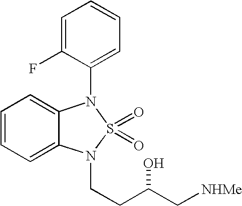
or pharmaceutically acceptable salt thereof.

13. The process of claim 2, wherein the reacting step is performed in water and Me-THF.

14. A process for the preparation of a compound of formula IA: 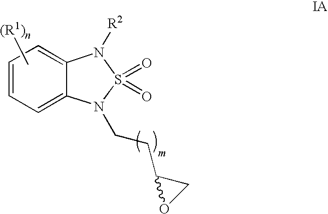
or a tautomer or salt thereof;
wherein:
m is an integer from 1 to 3;
n is an integer from 0 to 4;
R¹ is, independently at each occurrence, C₁-C₆alkyl, C₁-C₆alkoxy, halo, CF₃, OCF₃, hydroxy, C₁-C₅alkylC(O)O—, nitro, —CN, C₂-C₆alkenyl, C₂-C₆alkynyl, C₆-C₁₀aryl, C₄-C₁₀heteroaryl, C₁-C₆alkylS(O)—, C₁-C₆alkylS(O)₂—, C₁-C₆alkylS(O)₂NH—, C₁-C₆alkylS(O)₂N(C₁-C₆alkyl)-, C₆-C₁₀arylS(O)₂NH—, C₆-C₁₀arylS(O)₂N(C₁-C₆alkyl)- C₁-C₅alkylC(O)NH—, C₁-C₅alkylC(O)N(C₁-C₆alkyl)-, or C₆-C₁₀arylC(O)NH—, C₆-C₁₀arylC(O)N(C₁-C₆alkyl)-; wherein each C₆-C₁₀aryl or C₄-C₁₀heteroaryl is independently substituted with 0-3 C₁-C₆alkyl, C₁-C₆alkoxy, halo, CF₃, OCF₃, hydroxy, C₁-C₅alkylC(O)O—, nitro, —CN, C₂-C₆alkenyl, or C₂-C₆alkynyl groups; and each C₆-C₁₀arylS(O)₂NH—, C₆-C₁₀arylS(O)₂N(C₁-C₆alkyl)- or C₆-C₁₀arylC(O)NH—, C₆-C₁₀arylC(O)N(C₁-C₆alkyl)- is independently substituted with 0-3 C₁-C₆alkyl, C₁-C₆alkoxy, halo, CF₃, OCF₃, hydroxy, C₁-C₅alkylC(O)O—, nitro, —CN, C₂-C₆alkenyl, C₂-C₆alkynyl, C₁-C₆alkylS(O)—, C₁-C₆alkylS(O)₂—, C₁-C₆alkylS(O)₂NH—, C₁-C₆alkylS(O)₂N(C₁-C₆alkyl)-, C₁-C₅alkylC(O)NH—, or C₁-C₅alkylC(O)N(C₁-C₆alkyl)- groups;
R² is C₆-C₁₀aryl or C₄-C₁₀heteroaryl substituted with 0-5 C₁-C₆alkyl, C₁-C₆alkoxy, halo, CF₃, OCF₃, hydroxy, C₁-C₅alkylC(O)O—, nitro, —CN, C₂-C₆alkenyl, C₂-C₆alkynyl, C₁-C₆alkylS(O)—, C₁-C₆alkylS(O)₂—, C₁-C₆alkylS(O)₂NH—, C₁-C₆alkylS(O)₂N(C₁-C₆alkyl)-, C₆-C₁₀arylS(O)₂NH—, C₆-C₁₀arylS(O)₂N(C₁-C₆alkyl)-, C₁-C₅alkylC(O)NH—, C₁-C₅alkylC(O)N(C₁-C₆alkyl)-, C₆-C₁₀arylC(O)NH—, C₆-C₁₀arylC(O)N(C₁-C₆alkyl)-, or C₆-C₁₀aryl or C₄-C₁₀heteroaryl optionally substituted with C₁-C₆alkyl, C₁-C₆alkoxy, halo, CF₃, OCF₃, hydroxy, C₁-C₅alkylC(O)O—, nitro, —CN, C₂-C₆alkenyl, or C₂-C₆alkynyl; and [symbol]
represents an S-isomer, R-isomer or racemate;
the process comprising:
reacting a compound of formula IB: 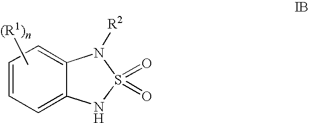
in the presence of a base with a compound of formula IC: 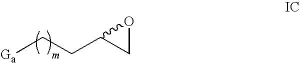
wherein Ga is an activating group.

15. The process of claim 14, wherein the base is potassium carbonate (K₂CO₃).

16. The process of claim 15, wherein the reacting step is also performed in the presence of tetrabutylammonium iodide (TBAI) and Me-THF.

17. The process of claim 14, wherein Ga is halo, tosylate, mesylate, or triflate.

18. The process of claim 17, wherein Ga is tosylate and the compound of formula IC is prepared by 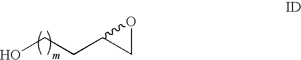
reacting tosyl chloride (TsCl) with a compound of formula ID in the presence of a base.

19. The process of claim 18, wherein the compound of formula ID is prepared by 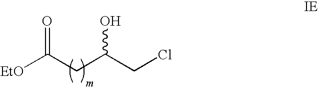
reacting a hydride and potassium phosphate (K₃PO₄) with a compound of formula IE.

20. The process of claim 14 wherein the compound of formula IB: 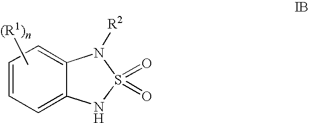
or a tautomer or salt thereof;
wherein:
n, R¹, and R² have the meaning given above;
is prepared by a process comprising protecting a compound of formula IF: 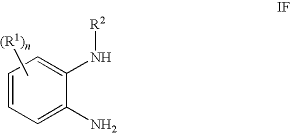
to form a compound of formula IG: 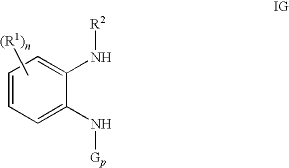
reacting SO(Ga)₂, wherein Ga is an activating group, with the compound of formula IG to form a compound of formula IH: 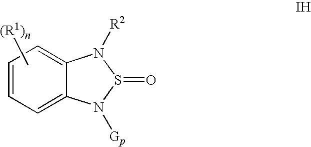
oxidizing the compound of formula IH to form a compound of formula IJ: 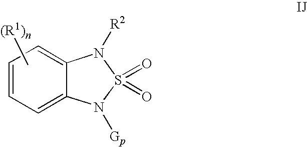
and deprotecting the compound of formula IJ to form the compound of formula IB.

21. The process of claim 20, wherein the Gp is selected from the group consisting of Boc, benzyl, acetyl, PMB, C₁-C₆alkyl, Fmoc, Cbz, trifluoroacetyl, tosyl and triphenylmethyl.

22. The process of claim 21, wherein Gp is Boc and the protecting step comprises reacting Boc anhydride (Boc₂O) with the compound of formula IF.

23. The process of claim 20, wherein Ga is Cl and the reacting step is performed in the presence of triethylamine (Et₃N).

24. The process of claim 20, wherein the oxidizing step is performed in the presence of ruthenium chloride (RuCl₃), sodium periodate (NaIO₄) and a biphasic toluene/water solution.

25. The process of claim 20, wherein the deprotecting step is performed in the presence of sodium methoxide (NaOMe) and toluene.

26. The process of claim 20, wherein the compound of formula IF is prepared by contacting R²—NH₂ with a compound of formula IK: 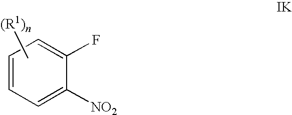
to form a compound of formula IL: 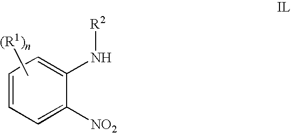
and hydrogenating the compound of formula IL to form the compound of formula IF.

27. The process of claim 26, wherein the contacting step is performed in the presence of potassium tertiary butoxide (t-BuOK).

28. The process of claim 26, wherein the hydrogenating step is performed in the presence of H₂ and palladium on carbon (Pd—C).

29. The process of claim 28, wherein the hydrogenating step comprises about 0.5% palladium on carbon (Pd—C).
